SEQUENCE LISTING (4 sequences)

SEQ ID NO: 1 - protein, 92 aa, Homo sapiens
APDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 2 - protein, 145 aa, Homo sapiens
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 3 - protein, 121 aa, Homo sapiens
SREPLRPWCHPINAILAVEKEGCPVCITVNTTICAGYCPTMMRVLQAVLPPLPQVVCTYRDVRFESIRLPGCPRGVDPVVSFPVALSCRCGPCRRSTSDCGGPKDHPLTCDHPQLSGLLFL

SEQ ID NO: 4 - protein, 24 aa, Homo sapiens
VPDGALRCPGPTAGLTRLSLTYLP

(19) United States
(12) Patent Application Publication
(10) Pub. No.: US 2024/0115662 A1
(43) Pub. Date: Apr. 11, 2024

(54) METHODS OF TREATING HYPOGONADOTROPIC HYPOGONADISM AND COGNITION IMPAIRMENT FOLLOWING A TRAUMATIC BRAIN INJURY

(71) Applicants: WISCONSIN ALUMNI RESEARCH FOUNDATION, Madison, WI (US); UNITED STATES GOVERNMENT AS REPRESENTED BY THE DEPARTMENT OF VETERANS AFFAIRS, Washington, DC (US)

(72) Inventor: Craig S. ATWOOD, Madison, WI (US)

(21) Appl. No.: 18/276,871

(22) PCT Filed: Mar. 2, 2022
(86) PCT No.: PCT/US2022/018517

§ 371 Date: Aug. 10, 2023

Related U.S. Application Data
(60) Provisional application No. 63/155,690, filed on Mar. 2, 2021.

(51) Int. Cl. A61K 38/24; A61K 31/565; A61K 31/567; A61K 31/5685; A61K 31/57; A61K 38/18; A61P 25/28

(52) U.S. Cl. CPC A61K 38/24; A61K 31/565; A61K 31/567; A61K 31/5685; A61K 31/57; A61K 38/1841; A61P 25/28

(57) ABSTRACT

Described herein are methods of inducing cognitive recovery of a subject suffering from a traumatic brain injury (TBI). The methods comprise administering to the subject pharmaceutical compositions including human chorionic gonadotropic (hCG) and/or luteinizing hormone (hLH), or a combination thereof.

### CROSS-REFERENCE TO RELATED APPLICATIONS

This application claims the benefit of priority to U.S. Provisional Patent Application No. 63/155,690 filed Mar. 2, 2021, which is hereby incorporated by reference, in its entirety for any and all purposes.

### STATEMENT OF GOVERNMENT SUPPORT

This invention was made with government support under grant number I21RX001371 awarded by VA Merit Review, grant number T32AG00213 awarded by National Institute on Aging, and grant number UL1T000427 awarded by the Institute for Clinical and Translational Research. The government has certain rights in the invention.

### TECHNICAL FIELD

The present technology relates generally to methods of inducing cognitive recovery of a subject suffering from a traumatic brain injury (TBI). The methods include administering to the subject an effective amount of human chorionic gonadotropic (hCG) and/or human luteinizing hormone (hLH), or a combination thereof.

### BACKGROUND

Traumatic brain injury (TBI) is a major public health problem due to the relatively high incidence rate (106 per 100,000 globally, (2) the lack of effective treatments. The incidence of TBI in males is 3 times that of females, but normalizes to 1:1 by age 65. The consequences of a TBI can include functional (e.g., decreased cognitive performance), psychopathological (e.g., post-traumatic stress disorder), neuroanatomical (e.g., cystic infarcts, neurodegeneration) and biochemical (e.g., inflammation changes).

An underappreciated endocrinological complication of TBI is hypogonadotropic hypogonadism (HH). TBI can markedly suppress pituitary gonadotropin secretion and gonadal sex steroid production. Such hypothalamic-pituitary-gonadal (HPG) axis hormones have well-described roles in the formation and maintenance of brain structure and cognitive function (reviewed in). TBI-induced HH is thought to result from damage to the hypothalamus or pituitary, and/or stress-induced cortisol-mediated suppression of the hypothalamic-pituitary-gonadal (HPG) axis. While prevalence rates for HH vary widely, likely due to the severity of the injury, location and type of injury, time of screening and design of the study, there is increasing consensus that even mild TBIs can induce HH and that severe TBIs induce persistent HH. This silent condition goes mostly undiagnosed and therefore untreated.

### SUMMARY OF THE PRESENT TECHNOLOGY

In one aspect, the present disclosure provides methods for inducing cognitive recovery of a subject suffering from a traumatic brain injury (TBI) comprising administering to the subject an effective amount of human chorionic gonadotropic (hCG), human luteinizing hormone (hLH), or a combination thereof.

In another aspect, the present technology provides a method for inducing cognitive recovery of a subject suffering from a traumatic brain injury (TBI) comprising: determining that the subject is capable of normal adult gonadal hormone synthesis and secretion; and administering to the subject an effective amount of human chorionic gonadotropic (hCG), human luteinizing hormone (hLH), or a combination thereof.

In yet another aspect, the present technology provides a method for inducing cognitive recovery of a subject suffering from a traumatic brain injury (TBI) comprising: determining that the subject is not capable of normal adult gonadal hormone synthesis or secretion (e.g., the subject is post-menopausal, andropausal, hypogonadal, and/or non-responsive to hCG-induced sex steroid production); and administering to the subject an effective amount of human chorionic gonadotropic (hCG), human luteinizing hormone (hLH), or a combination thereof, and an effective amount of a gonadal hormone, optionally wherein the gonadal hormone is selected from progesterone, estradiol, testosterone, inhibin B, AMH, or a combination of any two or more thereof.

### BRIEF DESCRIPTION OF THE DRAWINGS

FIGS. 1A-1D shows that hCG reverses controlled cortical impact (CCI)-induced decreases in circulating testosterone and progesterone. Plasma concentrations (mean±SEM) of testosterone (T) (FIG. 1A), progesterone (P₄) (FIG. 1B), 11-DOC (FIG. 1C) and corticosterone (FIG. 1D) in ng/mL on post-injury day (PID) −10, 1, 11, 19 and 29 for the following groups: Sham+vehicle (n=5), Sham+hCG (n=5), CCI+saline (n=8) and CCI+hCG (n=8). Data were analyzed using 2-way repeated measures ANOVA; post-hoc analyses were performed using the Tukey multiple comparison test (p<0.05; letters indicate differences between treatment groups and pre- and post-injury days).

FIGS. 2A-2D shows that RU-486 treatment attenuates hCG-induced reversal of circulating testosterone plasma concentrations. Plasma concentrations (mean±SEM) of T (FIG. 2A), P₄ (FIG. 2B), 11-DOC (FIG. 2C) and corticosterone (FIG. 2D) in ng/mL on PID-10, 1, 11, 19 and 29 for the following groups: RU-486: Sham+vehicle (n=5), RU-486: Sham+hCG (n=5), RU-486: CCI+saline (n=5) and RU-486: CCI+hCG (n=5). Data were analyzed using 2-way repeated measures ANOVA; post-hoc analyses were performed using the Tukey multiple comparison test (p<0.05; letters indicate differences between treatment groups and pre- and post-injury days). Differences between RU-486 induced changes in plasma hormones between treatment groups in FIGS. 1A-1D and FIGS. 2A-2D are illustrated by (1) vertical lines representing an increase in plasma hormone concentration in RU-486 treated animals, and (2) horizontal lines representing a decrease in plasma hormone concentration in RU-486 treated animals.

FIG. 3 shows that hCG signals hESC proliferation via LHCGR. hESCs grown in six-well plates coated with Matrigel in mTeSR1 media were treated for 6 days with (i) hCG (500 mIU/ml; lane A); (ii) increasing concentrations of the affinity-purified rabbit polyclonal antibody against amino acids 15 to 38 of the extracellular binding domain of LH/hCG receptor (1:1,000, 1:200, 1:100; lanes B, C, and D, respectively); and (iii) in combination with (500 mIU/ml of hCG (lanes E, F, and G). Cell number was counted by using the trypan blue assay. Results are expressed as mean±SEM, n=3; (*P<0.05; **P<0.005 compared with day 6 control). Above: A schematic of the LHCGR activation site and binding site of rabbit polyclonal antibody against amino acids 15 to 38 of the extracellular binding domain.

FIG. 4 shows that hCG induces hESC synthesis and secretion of P₄. The concentration of P₄ secreted into the media from hESCs treated ±hCG (500 mIU/ml) for 6 days. Results are expressed as micrograms P₄ per microgram cellular protein, mean±SEM, n=3; *P<0.001.

FIGS. 5A-5D show that P₄ differentiates hESC into neuroectoderm. FIG. 5A. hESC were grown in TESR1 media-Li in the presence of E₂ (10 nM), P₄ (2 μM) or E₂ (10 nM)+P₄ (2 μM) for 6 d and cell proliferation measured using the trypan blue assay. Results are expressed as mean±SEM, n=3 (*p<0.05, **p<0.005 compared to 6 d control). FIG. 5B. Equal amounts of protein from cell lysates of hESC treated for 9 d as described above were analyzed by immunoblot using a monoclonal antibody against nestin (clone 10C2; Chemicon, CA, USA). FIG. 5C. hESC were cultured for 4 days, enzymatically lifted and placed into EB media (containing serum) and rocked gently for an additional 4 days prior to being placed in one of two neural induction medias for an additional 11 days, where P₄ was either absence or present. During this transition from EB to rosettes, colony structures treated with P₄ also were treated with a Progesterone Receptor (PR) antagonist (RU-486; 20 μM) for 11 days. At 19 day, morphological observation and molecular analysis were performed. (i) Control structure, a minimum of three rosette structures were observed inside the cystic cavity (arrows). (ii) hESC structure in the absence of P₄ (no rosettes) (iii) PR antagonist treated rosettes (no rosettes; RU-486, 20 μM). FIG. 5D. Equal amounts of protein from cell lysates of above structures were analyzed by immunoblot for nestin.

FIG. 6A shows expression of LH receptor in primary hippocampal neurons. Neurons from rat embryonic day 18 hippocampi were differentiated in culture for 7 days and probed with the monoclonal anti-LH receptor antibody 3B5 (left) and stained with DAPI (right).

FIG. 6B shows that fluorescence micrographs of the forebrain SVZ show an increase in the number of BrdU-labeled cells in mice given subcutaneous infusions of LH compared with vehicle. Female mice (8-10-week old) were delivered LH subcutaneously for 2 days via osmotic pump (16 mg per day, 2 days). Modified from Mak G K, et al., Nat Neurosci 10: 1003-1011 (2007).

FIG. 7 shows that hCG restores CCI-induced decreases in circulating T and P₄. Plasma concentrations (mean±SEM) of testosterone and progesterone at PID-10, 1, 11, 19 and 29. Data were analyzed using 3-way repeated measures ANOVA; post-hoc analyses were performed using the Tukey multiple comparison test. Difference letters indicate significant differences among groups over days (P<0.05). Similar significant declines in circulating concentrations of T (74.5%) and P₄ (59.0%) were observed by PID1 for CCI injured animals (i.e., craniectomy+CCI+saline group). The hypogonadism induced by the surgery/CCI in both groups was maintained through day 29.

FIG. 8 shows that hCG improves performance in rotarod sensorimotor task. Rats were placed on a rotating rod accelerating at a constant rate (1 rotation per second per second) and allowed to run for up to 300 s. Time elapsed before fall was recorded (n=9-12/group, *p<0.05; letters indicate differences between groups and times).

FIG. 9 shows latency to reach MWM hidden platform during Acquisition Trials and Novel Platform Placement (NPP; Re-acquisition phase). Acquisition Phase (NE quadrant): CCI significantly increased latency to reach the platform, while hCG treatment of CCI animals reduced latency to reach the platform. Therefore, hCG reduced the detrimental effects of CCI on latency to reach the platform. Novel Platform Placement (SE quadrant): Sham/NSCon groups (vehicle and hCG) demonstrated increased latency to find the hidden platform after it was relocated to the SE quadrant, whereas latency to find the platform was not altered in CCI groups. This illustrates that while the Sham/NSCon groups retained memory of original platform location, CCI groups did not retain memory of platform location. Re-Acquisition Phase:_Sham/NSCon groups quickly learned the new location of the hidden platform, and while this learning was severely compromised in the CCI vehicle group, hCG enabled better learning of the new location of the hidden platform in CCI animals (n=9-12/group; p<0.05; a, b=differences between groups during acquisition phase; c, d=differences between groups during re-acquisition phase; e=differences within groups between the acquisition (PID10) and re-acquisition phases (PID20)).

FIG. 10 shows time spent in NE quadrant (after completing Acquisition Phase) and SE and NE quadrants (after Novel Platform Placement (NPP; Re-acquisition phase)). Probe Test 1 (NE quadrant): CCI significantly decreased the amount of time spent in the NE quadrant, an effect that was partially reversed by hCG treatment. Probe Test 2 (SE quadrant): Like in Probe Test 1, hCG treatment significantly increased time spent in the newer SE quadrant (platform placement 2 days prior) but not in the older NE quadrant (platform placement 12 days prior). Analysis of the accumulated time spent in both quadrants associated with the hidden platform indicated that compared to CCI animals treated with vehicle, hCG treatment improved the time spent in the platform associated quadrants to greater than chance (n=9-12/group; p<0.05; letters indicate differences between groups within each experimental condition).

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

The following terms are used throughout as defined below. All other terms and phrases used herein have their ordinary meanings as one of skill in the art would understand.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

“Drug” or “active agent” as used herein refers to any suitable therapeutic agent. Drugs/active agents for use in the present technology include human chorionic gonadotropic (hCG), human luteinizing hormone (hLH), 11β-[p-(Dimethylamino)phenyl]-17α-(1-propynyl)estra-4,9-dien-17β-ol-3-one (RU-486), or combinations of two more thereof. In any embodiment, the drugs/active agents for use in the present technology includes hCG.

“Effective amount” refers to the amount of compound (here, the drug) or composition required to produce a desired effect. Hence, an effective amount of a compound or composition of the present technology in the context of treatment (i.e., “a therapeutically effective amount”) refers to an amount of the compound or composition that alleviates, in whole or in part, symptoms associated with a disorder or disease (e.g., reverse or diminish the cognitive impairment/induce cognitive recovery), or slows or halts further progression or worsening of those symptoms. In the context of prevention, an effective amount prevents at least partially or provides prophylaxis for the disease or disorder in a subject at risk for developing the disease or disorder. One example of an effective amount includes amounts or dosages that yield acceptable toxicity and bioavailability levels for therapeutic (pharmaceutical) use. Determining a therapeutically effective amount of a compound described herein for treating a particular disorder or disease is well within the skill in the art in view of the present disclosure.

As used herein, a “subject” or “patient” is a mammal, such as a cat, dog, ungulate, rodent or primate. In any embodiments, the subject is a human. The term “subject” and “patient” can be used interchangeably.

“Treating” or “treatment” within the context of the present technology, means an alleviation, in whole or in part, of symptoms associated with a disorder or disease, or slowing, or halting of further progression or worsening of those symptoms. As a non-limiting example of treatment, a subject can be successfully treated for cognitive impairment induced by a TBI if, after receiving through administration an effective or therapeutically effective amount of one or more drugs or compositions described herein, the subject shows observable and/or measurable induced cognitive recovery. Treatment, as defined herein, of a subject, including a human being, is subject to medical aid with the object of improving the subject's condition, directly or indirectly. Treatment typically refers to the administration of an effective amount of a drug or composition containing a drug such as hCG as described herein.

“Traumatic brain injury” (“TBI”) as used herein includes brain damage resulting from any sudden, external, physical impact. The damage may be focal (confined to one area of the brain) or diffuse (happens in more than one area of the brain).

Pathology. The most common types of TBI are diffuse axonal injury, contusion, and subdural hemorrhage. Diffuse injury results when shearing, stretching, and/or angular forces pull on axons and small vessels. Impaired axonal transport leads to focal axonal swelling and may result in axonal disconnection after several hours. The most common locations are the corticomedullary (gray matter-white matter) junction (particularly in the frontal and temporal areas), internal capsule, deep gray matter, upper brainstem, and corpus callosum. Contusions occur when the brain moves within the skull enough to impact bone, causing bruising of the brain parenchyma (hemorrhage and edema). The most common locations for TBI are the superficial gray matter of the inferior, lateral and anterior aspects of the frontal and temporal lobes, with the occipital poles or cerebellum less often involved. Traumatic subdural hemorrhage occurs when the brain moves within the skull enough to tear the tributary surface veins that bridge from the brain surface to the dural venous sinus. The most common locations are the frontal and parietal convexities on the side of the injury; progression is common, with 25% demonstrating delayed hemorrhage in first 48 hours. Cerebral edema, swelling of the brain, breakdown of the blood-brain barrier and inflammation are common sequalae of TBI.

Neurodegeneration. Neuronal and glial cell death and traumatic axonal injury contribute to the overall pathology of TBI. In both head-injured humans and following experimental brain injury, dying neural cells exhibit either an apoptotic or a necrotic morphology. Apoptotic and necrotic neurons have been identified within contusions in the acute post-traumatic period, and in regions remote from the site of impact in the days and weeks after trauma, while degenerating oligodendrocytes and astrocytes have been observed within injured white matter tracts.

Symptoms/Complications. The severity of a brain injury may range from a mild concussion to a severe injury that results in coma. Symptoms and/or complications of TBI may comprise cognitive, behavioral, and/or motor deficits, including but are not limited to amnesia, inability to speak or understand language, mental confusion, difficulty concentrating, difficulty thinking and understanding, inability to create new memories, inability to recognize common things, abnormal laughing and crying, aggression, impulsivity, irritability, lack of restraint, persistent repetition of words or actions, dilated pupil, raccoon eyes, unequal pupils, blackout, dizziness, fainting, fatigue, difficulty speaking or slurred speech, persistent headache, a temporary moment of clarity, balance disorder, bleeding, bone fracture, bruising, depression, loss of smell, nerve injury, post-traumatic seizure, ringing in the ears, sensitivity to sound, and/or stiff muscles.

In one aspect, the present technology provides methods for inducing cognitive recovery of a subject (e.g., a human subject) suffering from a traumatic brain injury (TBI) including administering to the subject an effective amount of human chorionic gonadotropin (e.g., hCG), luteinizing hormone (e.g., hLH), or a combination thereof. Each of hCG and hLH may be naturally occurring or synthetic (including, e.g., recombinant, chemically synthesized or semi-synthetically prepared).

Naturally occurring hCG and hLH are heterodimeric glycoprotein hormones, each comprising an a subunit and a β subunit. Each subunit consists of a single polypeptide chain which are non-covalently bound to each other. The a subunit polypeptide is common to both hCG and LH, whereas the β subunits differ in sequence from each other. Each of the hormones may exist as a mixture of isoforms, including differentially glycosylated isoforms.

The amino acid sequence of the α subunit of hCG (92 aa), is identical to the sequence of the α subunit of LH, and is given below as SEQ ID NO:1:

```
                10 20 30 40 50 60
APDVQDCPEC TLQENPFFSQ PGAPILQCMG CCFSRAYPTP LRSKKTMLVQ KNVTSESTCC
                70 80 90
VAKSYNRVTV MGGFKVENHT ACHCSTCYYH KS
```

The alpha sub unit contains 2-N-linked glycosylation sites at amino acids 52 and 78.

The β subunit differs in sequence between hCG (145 aa) and LH (141 aa). The amino acid sequence of the β subunit of hCG is given below as SEQ NO:2:

```
                10 20 30 40 50 60
SKEPLRPRCR PINATLAVEK EGCPVCITVN TTICAGYCPT MTRVLQGVLP ALPQVVCNYR
                70 80 90 100 110 120
DVRFESIRLP GCPRGVNPVV SYAVALSCQC ALCRRSTTDC GGPKDHPLTC DDPRFQDSSS
              130 140
SKAPPPSLPS PSRLPGPSDT PILPQ
```

The β subunit of hCG contains 2-N linked glycosyiation sites at amino acids 13 and 30 and four O-linked glycosylation sites at amino acids 121, 127, 132 and 138.

The β subunit for LH (also known as lutropin) has the amino acid sequence of SEQ ID NO: 3:

```
                10 20 30 40 50 60
SREPLRPWCH PINAILAVEK EGCPVCITVN TTICAGYCPT MMRVLQAVLP PLPQVVCTYR
                70 80 90 100 110 120
DVRFESIRLP GCPRGVDPVV SFPVALSCRC GPCRRSTSDC GGPKDHPLTC DHPQLSGLLF
L
```

The β subunit of LH is only N-glycosylated at position 30 for a total of three glycosylated residues in the hormone. By comparison, hCG has a total of eight glycosylation sites. The additional amino acids in the beta subunit and the overall heavier glycosylation of hCG results in a half-life of about 36 hours, whereas the half-life of LH is about 20 minutes. Alternatively, the terminal half-life via subcutaneous administration is about 32-33 hours for recombinant hCG vs about 21-24 hours for r-hLH.

Various isoforms of the hCG and hLH described herein may be used in the present methods. Such isoforms may include amino acid substitutions, including conservative substitutions, insertions or deletions. In any embodiments, the α subunit of hCG or hLH employed in the present methods may have substantial sequence identity to SEQ ID NO: 1, including, e.g., at least 85%, 86%, 87%, 88%, 89%, 90%, 91%, 92%, 93%, 94%, 95%, 96%, 97%, 98%, 99%, or 100% sequence identity or a range between and including any two of the foregoing values. In any embodiments, the β subunit of hCG employed in the present methods may have substantial sequence identity to SEQ ID NO: 2, including, e.g., at least 85%, 86%, 87%, 88%, 89%, 90%, 91%, 92%, 93%, 94%, 95%, 96%, 97%, 98%, 99%, or 100% sequence identity or a range between and including any two of the foregoing values. In any embodiments, the β subunit of LH employed in the present methods may have substantial sequence identity to SEQ ID NO: 3, including, e.g., at least 85%, 86%, 87%, 88%, 89%, 90%, 91%, 92%, 93%, 94%, 95%, 96%, 97%, 98%, 99%, or 100% sequence identity or a range between and including any two of the foregoing values. In any embodiment, the hCG and/or hLH may be any known glycoform, including, e.g., hyperglycosylated hCG, with any known glycosylation pattern in any of the N-glycan and/or O-glycans.

In certain embodiment, where the subject is a non-human mammal, the CG and/or LH polypeptides used in the present methods refer to the CG and/or LH isoforms native to the subject species and may include deletional, insertional, or substitutional mutants of such native CG and/or LH. In any embodiments, the CG and/or LH may also be a functional agonist of a native mammalian hCG/LH receptor.

In any embodiments, administering to the subject an effective amount of hCG, hLH, or a combination thereof may be effective to prevent, reduce, ameliorate, or eliminate one or more of deficits and/or symptoms caused by TBI, including but not limited to any of those disclosed herein. In any embodiments, the administering induces cognitive recovery in the subject from any of the cognitive, behavioral, and/or motor deficits listed above. In any embodiments, the administering improves vestibular balance and motor coordination, medium-term memory and/or long-term memory, and/or reduces gross lesion size, as compared to the subject before the administration or subjects suffering from a traumatic brain injury (TBI) of similar nature and degree but without the administration.

For the purposes of the disclosed methods, the subject may achieve at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more improvements in any human neurological tests. Examples of those tests include but are not limited to imaging tests (e.g., CT and MM) and Glasgow Coma Scale (GCS) test. Other examples may include speech and language tests, social communication skills tests, tests of swallowing abilities, tests of breathing abilities and lung function, any cognition tests or questions to see how the patient's thinking, reasoning, problem-solving, understanding, and remembering abilities, and blood tests to detects two proteins UCH-L1 and GFAP which are released by the brain into the bloodstream when a mild concussion occurs. See J Neurotrauma. 27(6): 983-989 (2010); www.sralab.org/rehabilitation-measures/neurological-outcome-scale-traumatic-brain-injury; and www.nichd.nih.gov/health/topics/tbi/conditioninfo/diagnose. Neuropsychological assessments to learn more about the patient's brain and social functions, including the ability to control one's behavior and actions, may also be applied. Those functional tests are well-known to a person of ordinary skill in the art.

In any embodiments, the subject may further suffer from a TBI-associated impairment comprising hypogonadotropic hypogonadism (HH). HH may be a subclinical condition, identified only by hormonal tests, or its clinical manifestations may be acute and severe. Clinic tests to identify HH are well-known to a person of ordinary skill in the art.

In any embodiments, TBI or (TBI)-associated impairment is induced by an injury to a hypothalamus or pituitary gland of the subject. In any embodiments, the TBI or (TBI)-associated impairment is induced by the suppression of the synthesis and secretion into the bloodstream of hormones produced by the hypothalamic-pituitary-gonadal (HPG) axis.

In any embodiments, the hCG, hLH, or combination thereof is administered about 2 days to about 7 days per week. In any embodiments, the hCG, hLH, or combination thereof is administered 2 days, 3 days, 4 days, 5 days, 6 days, or about 7 days per week.

In any embodiments, the hCG, hLH, or combination thereof is administered every other day, every other 2 days, or every other 3 days.

In any embodiments, the hCG, hLH, or combination thereof is administered for about 1 week to about 6 weeks. In any embodiments, the hCG, hLH, or combination thereof is administered for about 1 week, 2 weeks, 3 weeks, 4 weeks, 5 weeks, or 6 weeks.

In any embodiments, the hCG, hLH, or combination thereof is administered for about 1 months to about 18 months. In any embodiments, the hCG, hLH, or combination thereof is administered for about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, or about 18 months.

In any embodiments, an effective amount of the hCG, hLH, or combination thereof administered comprises about 0.1 IU/kg to about 1000 IU/kg of the hCG, hLH, or combination thereof. In any embodiments, an effective amount of the hCG, hLH, or combination thereof administered comprises about 0.1 IU/kg to about 100 IU/kg, about 100 IU/kg to about 200 IU/kg, about 200 IU/kg to about 300 IU/kg, about 300 IU/kg to about 400 IU/kg, about 400 IU/kg to about 500 IU/kg, about 500 IU/kg to about 600 IU/kg, about 600 IU/kg to about 700 IU/kg, about 700 IU/kg to about 800 IU/kg, about 800 IU/kg to about 900 IU/kg, or about 900 IU/kg to about 1000 IU/kg of the hCG, hLH, or combination thereof. In any embodiments, an effective amount of the hCG, hLH, or combination thereof administered comprises about 1 IU/kg to about 5 IU/kg, about 5 IU/kg to about 10 IU/kg, about 10 IU/kg to about 15 IU/kg, about 15 IU/kg to about 20 IU/kg, about 20 IU/kg to about 25 IU/kg, about 25 IU/kg to about 30 IU/kg, about 30 IU/kg to about 35 IU/kg, about 35 IU/kg to about 40 IU/kg, about 40 IU/kg to about 45 IU/kg, about 45 IU/kg to about 50 IU/kg, about 50 IU/kg to about 55 IU/kg, about 55 IU/kg to about 60 IU/kg, about 60 IU/kg to about 65 IU/kg, about 65 IU/kg to about 70 IU/kg, about 70 IU/kg to about 75 IU/kg, about 75 IU/kg to about 80 IU/kg, about 80 IU/kg to about 85 IU/kg, about 85 IU/kg to about 90 IU/kg, about 90 IU/kg to about 95 IU/kg, about 95 IU/kg to about 100 IU/kg, about 100 IU/kg to about 115 IU/kg, about 115 IU/kg to about 120 IU/kg, about 120 IU/kg to about 125 IU/kg, about 125 IU/kg to about 130 IU/kg, about 130 IU/kg to about 135 IU/kg, about 135 IU/kg to about 140 IU/kg, about 140 IU/kg to about 145 IU/kg, about 145 IU/kg to about 150 IU/kg, about 150 IU/kg to about 155 IU/kg, about 155 IU/kg to about 160 IU/kg, about 160 IU/kg to about 165 IU/kg, about 165 IU/kg to about 170 IU/kg, about 170 IU/kg to about 175 IU/kg, about 175 IU/kg to about 180 IU/kg, about 180 IU/kg to about 185 IU/kg, about 185 IU/kg to about 190 IU/kg, or about 190 IU/kg to about 200 IU/kg of the hCG, hLH, or combination thereof.

In any embodiments, the method may further comprises administering to the subject an effective amount of 11β-[p-(Dimethylamino)phenyl]-17α-(1-propynyl)estra-4,9-dien-170-ol-3-one (RU-486).

In any embodiments, the subject is capable of normal adult gonadal hormone synthesis and secretion. As used herein, “gonadal hormones” refer to hormones produced by the gonads, and include sex steroid and protein (or peptide) hormones. Sex steroids include but are not limited to estradiol (E₂), progesterone (P₄), and testosterone. Gonadal protein (or peptide) hormones include but are not limited to inhibin B and Anti-Müllerian hormone (AMH). Gonadal hormones generally exert their effects via nuclear receptors, but can also work via membrane receptors such as GPCRs. In any embodiments, the gonadal hormone comprises progesterone, estradiol, testosterone, inhibin B, Anti-Müllerian hormone (AMH), or a combination of any two or more thereof. In any embodiments, the subject is a human. In any embodiments, the human is a pre-menopausal female or a pre-andropausal male, optionally wherein the human is a 15- to 45-year-old female or 16- to 50-year-old male. For any subject of neonates through to the time of puberty (e.g., females less than 15 year old, or males less than 16 year old), the hCG, hLH, or combination thereof may be administered with caution that hCG might drive pubertal growth in children because gonadal sex hormone production may occur as a results of the administration.

In any embodiments, normal adult gonadal hormone synthesis and secretion in the subject may be determined by normal concentrations of one or more of gonadal hormones in the circulation (i.e., blood) in the subject. As non-limiting references, the following table (Table A) summarizes the normal concentrations of gonadal hormones in circulation of adult males or females capable of normal adult gonadal hormone synthesis and secretion, and the concentrations of gonadal hormones of post-menopause females an example of subjects not capable of normal adult gonadal hormone synthesis or secretion).

TABLE A

| Hormone | Men (adult) | | Women (adult) | | Women, Post-menopause | |
|---|---|---|---|---|---|---|
| Testosterone | 240-950 | ng/dL | 8-60 | ng/dL | | |
| Estradiol | 10-80 | pg/mL | 15-350 | pg/mL | <5-40 | pg/mL |
| Progesterone | <0.20 | ng/mL | 0.06-24 | ng/mL | <0.05-0.13 | ng/mL |
| AMH | <13 | ng/mL | 0.6-12 | ng/mL | <0.9 | ng/mL |
| Inhibin B | 47-383 | pg/mL | <80-224 | pg/mL | <12 | pg/mL |

Levels of gonadal hormones in adult women vary, depending on the stage of the menstrual cycle. Variations of gonadal hormone levels in men may also occur due to circadian rhythms, but may vary less than those caused by the menstrual cycle in women.

In some embodiments, the subject is not capable of normal adult gonadal hormone synthesis or secretion, and wherein the method further comprises separately, simultaneously, or subsequently administering to the subject an effective amount of a gonadal hormone. In any embodiments, the gonadal hormone is selected from progesterone, estradiol, testosterone, inhibin B, Anti-Müllerian hormone (AMH), or a combination of any two or more thereof.

In another aspect, the present technology provides a method for inducing cognitive recovery of a subject suffering from a traumatic brain injury (TBI) comprising: determining that the subject is capable of normal adult gonadal hormone synthesis and secretion; and administering to the subject an effective amount of human chorionic gonadotropic (hCG), human luteinizing hormone (hLH), or a combination thereof.

In yet another aspect, the present technology provides a method for inducing cognitive recovery of a subject suffering from a traumatic brain injury (TBI) comprising: determining that the subject is not capable of normal adult gonadal hormone synthesis or secretion; and administering to the subject an effective amount of human chorionic gonadotropic (hCG), human luteinizing hormone (hLH), or a combination thereof, and an effective amount of a gonadal hormone. In any embodiments, the gonadal hormone comprises progesterone, estradiol, testosterone, inhibin B, Anti-Müllierian hormone (AMH), or a combination of any two or more thereof.

In any embodiments, the step of determining whether the subject is or is not capable of normal adult gonadal hormone synthesis or secretion may include a two-step assessment. Step (1) is a high level assessment including assessing the patients age and reproductive status by determining whether the subject is pre-menopausal and pre-andropausal, is normally menstruating, exhibits sex steroid levels in the blood stream indicative of reproductive concentrations in women and men, and/or is hypogonadal. Step (2): if it is unclear in step (1), the subject could be tested to see if the subject is or is not responsive to hCG treatment by measuring HCG-induced sex steroid production. For example, after 1 to 24 hours of injecting a one off dose of hCG (10-1000 IU) to the subject, sex steroid concentrations in the bloodstream may be measured. A quickly increased circulating sex steroid concentrations would indicate that the subject is capable of sex steroid production. For example, a 500 IU dose of hCG generally would increases serum testosterone by greater than 50% within 24-48 hours in a male subject capable of normal adult gonadal hormone synthesis or secretion, or greater than 50% increase in estradiol or progesterone in a female subject capable of normal adult gonadal hormone synthesis and secretion. If a 500 IU dose of hCG does not increases serum testosterone by greater than 50% within 24-48 hours in a male subject, or greater than 50% increase in estradiol or progesterone in a female subject, the male or female subject may be considered not capable of normal adult gonadal hormone synthesis or secretion.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydrobromic acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺) ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

The present technology provides pharmaceutical compositions and medicaments comprising any one of the embodiments of the drugs disclosed herein and one or more pharmaceutically acceptable carriers or excipients. The compositions may be used in the methods and treatments described herein. The pharmaceutical composition may include an effective amount of any of one of the embodiments of the compositions disclosed herein for inducing cognitive recovery of a subject suffering from a traumatic brain injury (TBI).

The compositions described herein can be formulated for various routes of administration, for example, by parenteral, rectal, nasal, vaginal administration, or via implanted reservoir. Parenteral or systemic administration includes, but is not limited to, subcutaneous, intravenous, intraperitoneal, and intramuscular injections. The following dosage forms are given by way of example and should not be construed as limiting the instant present technology.

Injectable dosage forms generally include solutions or aqueous suspensions which may be prepared using a suitable dispersant or wetting agent and a suspending agent so long as such agents do not interfere with the activity of the drugs described herein. Injectable forms may be prepared with acceptable solvents or vehicles including, but not limited to sterilized water, Ringer's solution, 5% dextrose, or an isotonic aqueous saline solution. In any embodiment, the hCG may be in the form of Pregnyl® (hCG, 400 IU/kg; Merck & Co., Inc., Whitehouse Station, NJ).

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference. Thus, the present technology provides a pharmaceutical composition comprising any drug as described herein and a pharmaceutically acceptable carrier or excipient.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology. By way of example only, such dosages may be used to administer effective amounts of the cationic peptide drug(s) to the patient and may include about 0.1 mg/kg, about 0.2 mg/kg, about 0.3 mg/kg, about 0.4 mg/kg, about 0.5 mg/kg, about 0.75 mg/kg, about 1 mg/kg, about 1.25 mg/kg, about 1.5 mg/kg, or a range between and including any two of the forgoing values. Such amounts may be administered parenterally as described herein and may take place over a period of time including but not limited to 5 minutes, 10 minutes, 20 minutes, 30 minutes, 45 minutes, 1 hour, 2 hours, 3 hours, 5 hours, 10 hours, 12, hours, 15 hours, 20 hours, 24 hours or a range between and including any of the foregoing values. The frequency of administration may vary, for example, once or twice per day, per 2 days, per 3 days, per week, per 10 days, per 2 weeks, every other day, or a range between and including any of the foregoing frequencies. Alternatively, the compositions may be administered once per day on 2, 3, 4, 5, 6 or 7 consecutive days. A complete regimen may thus be completed in only a few days or over the course of 1, 2, 3, 4, 5, 6 or more weeks.

### EXAMPLES

The examples herein are provided to illustrate advantages of the present technology and to further assist a person of ordinary skill in the art with preparing or using the drugs of the present technology. To the extent that the compositions include ionizable components, salts such as pharmaceutically acceptable salts of such components may also be used. The examples herein are also presented in order to more fully illustrate certain aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### Materials and Methods

### Subjects

Male Sprague Dawley (SD) rats (n=58, 5-6-months old) were acquired from Harlan Laboratories Inc. (Madison, WI) and acclimated to the environment over 2 days. Rats were then weighed and handled for no less than 5 min each for 5 consecutive days and daily thereafter while being housed, fed and maintained on a 12-hour reverse light/dark cycle. The Institutional Animal Care and Use Committee (Animal Component of Research Protocol) at the William S. Middleton Veterans Administration Hospital approved the procedures used in this study and the research was conducted in an AAALAC-approved facility. Experimenters were blinded as to the identity of the animals throughout injections, blood collections, and body weight and hormone data analyses.

### Surgeries

All surgical procedures were carried out under isoflurane gas anesthesia (5% for induction; 1.5-3.0% for maintenance, craniectomy ˜15-30 min duration; craniectomy+CCI injury ˜25-45 min duration). An anesthesia chamber was used for induction and a nose cone was used for maintenance.

### Controlled Cortical Impact (CCI) and Sham Surgeries.

Anesthetized rats were mounted in a Kopf stereotaxic device (Model 900; Tujunga, CA), where the animal's head was held in place by non-traumatic ear bars and a bite bar. Anesthesia was maintained by nose cone while the head was shaved and sterilized with 70% ethanol and Betadine™ (Purdue Products L.P., Stamford, CT) antiseptic solution. Throughout surgery anesthesia levels were monitored closely and were frequently adjusted as need, based on heart rate, respiration rate and oxygen saturation. A homeothermic blanket control unit (Harvard Apparatus, Holliston, MA) was used to monitor body temperature and to prevent hypothermia throughout surgery.

Under aseptic conditions, the cranium and its bony landmarks including bregma (β) and lambda (λ) were exposed by making a midline incision along the scalp into the skin and fascia covering the skull. A 6 mm diameter craniectomy was centered on the midline at 2.5 mm anterior to β. The cortical impact was made at 2.5 mm anterior to β over the midline of the medial frontal cortex with an Impact One™ Stereotaxic CCI instrument (Leica, Buffalo Grove, IL), using a 5 mm impactor (bit size), traveling at 2.25 m/s (velocity), extending 3 mm below the cortical surface (impact depth) for 100 ms (dwell time). Sham-injured groups received the same surgical procedures up to and including craniectomy but no CCI injury. After surgery, the rats were placed on a heating pad, monitored closely and upon awakening were tested 30 min later for righting reflex to assess any immediate effects of craniectomy or CCI injury on righting ability, and then returned to their home cages.

### Experimental Design

Once out of quarantine all rats were weighed and handled for one week. The final body weights at the end of this week were (1) ranked from highest to lowest (as a function of age) and then (2) used to assigned each rat to a Surgery/Treatment group in a counterbalanced manner (i.e., using the ABBA method).

Experiment 1: Rats were assigned to the following groups: Sham+saline (n=5), Sham+hCG (n=5), CCI+saline (n=8), CCI+hCG (n=8). Baseline blood draws were collected from all animals. Beginning 1-hour after craniectomy or CCI, Pregnyl® (hCG, 400 IU/kg; Merck & Co., Inc., Whitehouse Station, NJ) or saline (0.9% NaCl in deionized H₂O; equivalent volume to that injected for hCG) was injected intramuscularly every other day for 29 days. Pregnyl® is a highly purified pyrogen-free preparation obtained from the urine of pregnant females. Each vial contains 10,000 USP units of sterile dried powder with 5 mg monobasic sodium phosphate and 4.4 mg dibasic sodium phosphate that is diluted in solvent containing water, 0.56% sodium chloride and 0.9% benzyl alcohol (www.drugs.com/pro/pregnyl.html).

Experiment 2: Rats were assigned to the following groups: Sham+saline+RU-486 (n=5), Sham+hCG+RU-486 (n=5), CCI+saline+RU-486 (n=5), or CCI+hCG+RU-486 (n=5). RU-486 (Mifepristone, 100 mg/mL solution, CAS Number 84371-65-3; 40 mg/kg in 100% ethanol; Sigma-Aldrich Corp., St. Louis, MO,) was injected intraperitoneallyl 5-20 min before every hCG or saline control injection.

### Blood Collection and Hormone Analyses

Rats were anesthetized (between 9:00 a.m.-12:00 noon) and their tails placed in a 200 mL beaker filled with warm water (≤44° C.) for 5 min. The tail was clean with 70% alcohol, the minimal amount of the tail tip snipped with a blade, and/or the wound reopened by removal of the scab for subsequent bleeds, and ˜1 mL of whole blood was collected directly into EDTA tubes at baseline (PID-10) and at PID, 1, 11, 19 and 29. Blood collected did not exceed 1% of body weight every 2-week period. Animals were injected with Lactate Ringers solution (5 mL) for fluid resuscitation. At the terminal bleed (day 29), blood also was collected via heart puncture. Collected blood was immediately centrifuged at 4,000 g for 10-20 min and the plasma aliquoted into Eppendorf tubes for storage at −80° C. Plasma samples were analyzed at the Assay Services Laboratory in the Wisconsin National Primate Research Center of the UW-Madison Institute for Clinical and Translational Research for progesterone (P₄), T, 11-deoxycorticosterone (11-DOC) and corticosterone adapted from a method as described in Kenealy B P, et al., J Neurosci. 33(49): 19051-19059 (2013), and in Kenealy B P, et al., Endocrinology 157(1): 70-76 (2016), both of which are hereby incorporated by reference in their entireties. Briefly, to plasma samples (400 μl) internal standard (200 pg d9-progesterone and d5-testosterone and 1 ng d4-cortisol) was added and the samples were extracted with methyl tert butyl ether. The organic phase was transferred to a clean vial and evaporated to dryness and then a second dichloromethane extraction was performed. The organic phase was transferred into a clean test tube and evaporated to dryness and reconstituted in mobile phase. Samples were analyzed on a QTRAP 5500 quadruple linear ion trap mass spectrometer (AB Sciex, Framingham, MA) equipped with an atmospheric pressure chemical ionization source. The system includes two Shimadzu LC20ADXR pumps and a Shimadzu SIL20ACXR autosampler (Addison, IL). A sample of 30 μl was injected onto a Phenomenex Kinetex 2.6u C18 100 A, 100×2.1 mm column (Torrance, CA) for separation using a mobile phase: water with 1% formic acid (Solution A) and acetonitrile with 1% formic acid (Solution B), at a flow rate of 200 μl/min. Quantitative results were recorded as multiple reaction monitoring (MRM) area counts after determination for the response factor for each compound and internal standard. Each steroid had a MRM used for quantitation and 1 or 2 additional MRMs as qualifiers. The linearity was r>0.818 and the curve fit was linear with 1/x weighting. None of the compounds of interest were detected in blank or double blank samples. Inter-assay coefficient's of variation were determined from a pool of rat plasma: T—2.1%, P₄—11.2%, 11-DOC—5.3%, corticosterone—8.0%, androstenedione—13.3%.

### Statistical Analysis

A mixed factorial analysis of variance (ANOVA) for repeated measures was performed on the weight, behavioral, hormonal and gross lesion data (GraphPad Prism, v.7; GraphPad Software, Inc., La Jolla, CA). Post-hoc analyses were performed using the Tukey multiple comparison test. Independent paired t-tests were also used to compare the differences between baseline (pre-injury) and post-injury data when data were normally distributed. Hormone data collected on post-surgery (PSD) or post-injury (PID) days were analyzed using the R statistical program, V.3.4.1 (R: A language and environment for statistical computing (program), Vienna, Austria: R Foundation for Statistical Computing, 2008), with package ‘rmcorr’ (rmcorr: Repeated Measures Correlation (program), R package version 0.2.0, 2017), based on statistical technique as described in Bland J M and Altman D G., Bmj. 310(6977): 446 (1995); and in Bakdash J Z and Marusich L R., Front Psychol. 8: 456 (2017), both of which are hereby incorporated by reference in their entireties. Non-repeated baseline data were analyzed using Pearson's correlation with the R program. Statistical significance was established at p≤0.05.

### Example 1: Craniectomy and Controlled Cortical Impact Injury Induce Hypogonadism and Hypoadrenalism

ANOVA indicated a significant main effect of treatment for T (F(3,82)=14.35, p<0.0001), P₄ (F(3,80)=11.34, p<0.0001), 11-DOC (F(3,88)=9.27, p<0.0001), and corticosterone (F(3,88)=24.98, p<0.0001). A main effect of day was found for P₄ (F(3,80)=5.26, p=0.0023), but not for T (F(3,82)=1.02, p=0.386), 11-DOC (F(3,88)=1.31, p=0.277), and corticosterone (F(3,88)=0.91, p=0.440). No main effects of treatment×day interaction was found for T (F(9,82)=0.33, p=0.964), P₄ (F(9,80)=1.74, p=0.095), 11-DOC (F(9,88)=1.07, p=0.396), or corticosterone (F(9,88)=0.94, p=0.498).

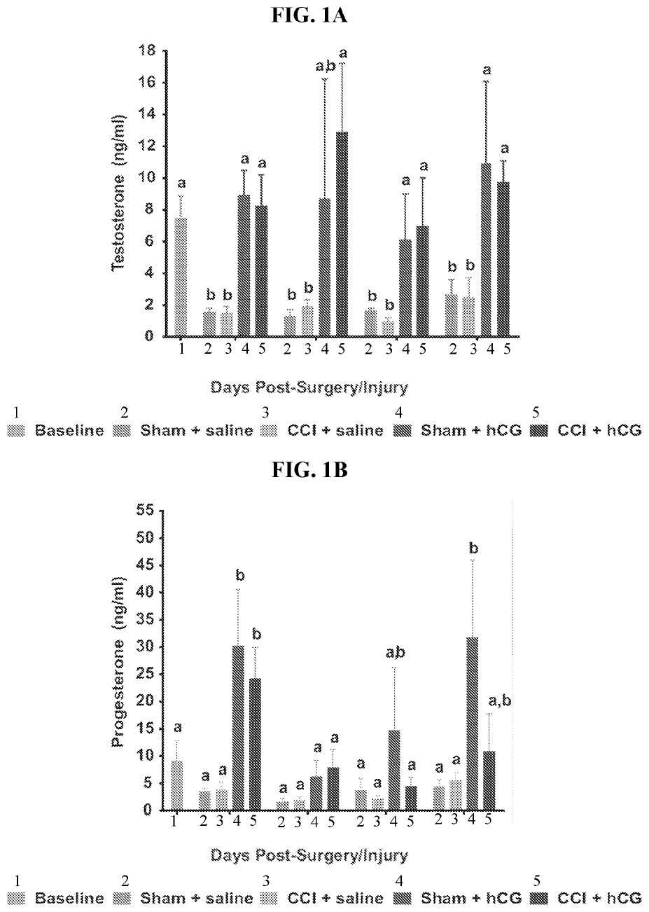

Sham surgery (craniectomy+saline group) in male adult rats induced a decline from baseline in the circulating concentrations of T (79.1%; 7.5±1.5 ng/mL to 1.6±0.3 ng/mL; p<0.05), P₄ (61.6%; 9.0±3.8 ng/mL to 3.5±0.7 ng/mL; p=0.061), 11-DOC (46.6%; 338.3±55.8 ng/mL to 180.7±3.3 ng/mL, p<0.05) and corticosterone (56.2%; 218.7±24.5 ng/mL to 95.9±2.2 ng/mL, p<0.05) by PSD1 (FIGS. 1A-1D) (see Geddes R I, et al., PLoS One. 12(1): e0169494 (2017), which is hereby incorporated by reference in its entirety). Similar declines in circulating concentrations of T (80.0%, 1.5±0.4 ng/mL; p<0.01), P₄ (56.8%, 3.9±1.4 ng/mL; p=0.065), 11-DOC (48.4%, 174.5±19.4 ng/mL; p<0.05) and corticosterone (32.5%, 147.7±17.4 ng/mL; p<0.05) were observed by PID 1 for CCI injured animals (i.e., craniectomy+CCI+saline group), indicating that Sham surgery alone was sufficient to induce hypogonadotropic hypogonadism (FIGS. 1A and 1B) and hypoadrenalism (FIGS. 1C and 1D) (see Geddes R I, et al., PLoS One. 12(1): e0169494 (2017)). Circulating concentrations for all hormones in both Sham surgery and CCI injured animals remained at these lower concentrations through PSD/PID 29 (except corticosterone in the CCI group on PID 29, which rose to 190.0±33.0 ng/mL, FIG. 1D). Circulating concentrations of androstenedione did not significantly change from baseline (0.58±0.10 ng/mL) in Sham surgery or CCI injury groups (data not shown). These results suggest that sham surgery, and sham surgery plus a bilateral moderate-to-severe CCI injury, induces hypogonadism in rats.

### Example 2: hCG Reverses Craniectomy and CCI-induced Hypogonadism and Attenuates Hypoadrenalism

hCG treatment of animals that underwent a craniectomy (Sham surgery) or craniectomy plus CCI injury (CCI group) significantly increased circulating concentrations of T and P₄ back to baseline concentrations by PSD/PID 1 (FIGS. 1A and 1B). Unlike P₄, elevations in T were maintained through PSD/PID 29. While a significant main effect of treatment group for androstenedione concentration also was identified, post-hoc analyses determined that androstenedione concentration was only elevated on PID 11 in the CCI+hCG group (4.5±2.1 ng/mL) compared to the CCI+saline group (0.51±0.2 ng/mL, p<0.03). hCG treatment transiently reduced circulating 11-DOC in Sham surgery animals (123.1±24.6 ng/mL) when compared to Sham+saline animals (180.7±3.3 ng/mL; p<0.05) on PID 1, but not on PID 11, 19 and 29 (FIG. 1C). hCG treatment had no effect on increasing corticosterone concentrations in Sham animals at any time point, but did increase circulating corticosterone in the CCI animals on PID 1 and 11 (FIG. 1D). Together, these results demonstrate that hCG can reverse hypogonadism induced by a craniectomy or a craniectomy+CCI injury, but has lesser effect on reversing hypoadrenalism.

### Example 3: RU-486 Diminishes Craniectomy+CCI-induced Hypoadrenalism

ANOVA indicated a significant main effect of treatment for T (F(3,71)=3.76, p=0.0145), P₄ (F(3,61)=5.82, p=0.0011), 11-DOC (F(3,61)=3.66, p=0.0171), and corticosterone (F(3,61)=5.41, p=0.0023). A main effect of day was found for P₄ (F(3,61)=5.82, p=0.0015) and corticosterone (F(3,61)=3.38, p=0.0238), but not for T (F(3,71)=1.86, p=0.145) or 11-DOC (F(3,61)=1.80, p=0.158). No main effects of treatment×day interaction was found for T (F(9,71)=0.33, p=0.965), P₄ (F(9,61)=1.09, p=0.3838), 11-DOC (F(9.61)=0.54, p=0.836), or corticosterone (F(9,61)=0.65, p=0.750).

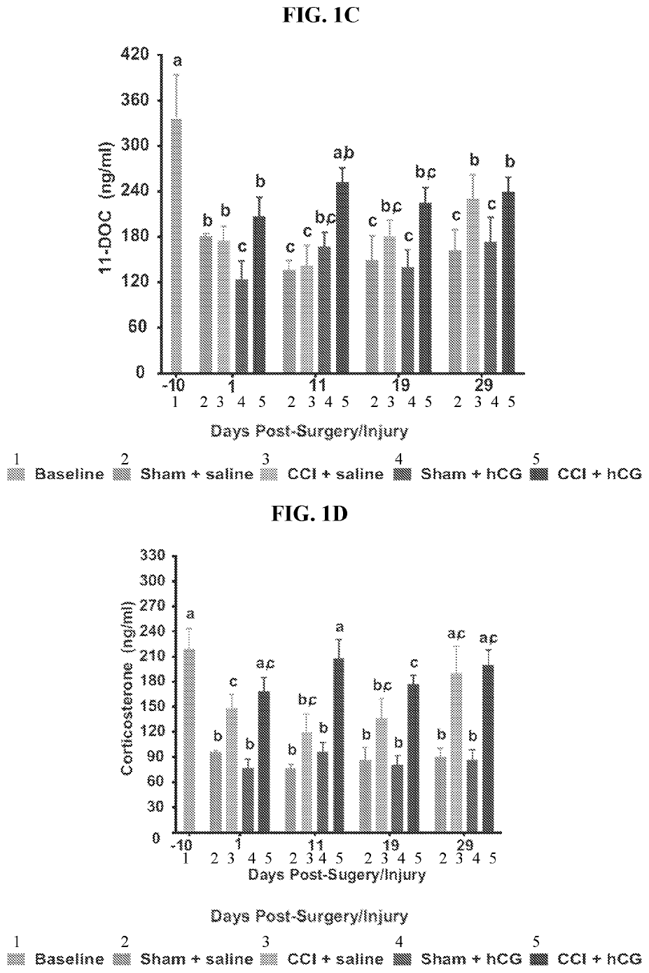

Pretreatment of animals with RU486, a P₄ receptor and glucocorticoid receptor antagonist, had little effect on Sham+hCG animals suppressing only P₄ concentration on PID 29 (8.5±4.5 ng/mL vs. 31.8±14.2 ng/mL, p<0.05; compare FIGS. 1B and 2B). In CCI injured animals, RU-486 pretreatment increased P₄ concentrations on PID 1 (40.9±10.0 ng/mL vs. 24.2±5.8 ng/mL, p<0.05), and suppressed T concentrations on PID 11 (4.5±1.5 ng/mL vs. 12.9±4.3 ng/mL, p<0.05) and PID 19 (3.1±1.1 ng/mL vs. 9.7±1.4 ng/mL, p<0.05; FIGS. 2A and 2B). RU486 pretreatment had more significant effects on 11-DOC and corticosterone; preventing the Sham+saline treatment-induced decrease in 11-DOC through PID29 (FIG. 2C), and preventing in Sham+hCG rats the decrease in 11-DOC at PID 1 (282.4±28.0 ng/mL vs. 123.1±24.6 ng/mL, p<0.05). RU486 pretreatment prevented Sham surgery-induced decreases in corticosterone through PID 29 in both saline and hCG-treated animals (except on PID 19 in the Sham+hCG group; FIG. 2D). RU486 pretreatment had no significant effects on circulating 11-DOC and corticosterone in CCI injured animals (FIGS. 2C and 2D).

### Example 4: Relationships Between Circulating Steroid Concentrations Before and After Sham Surgery, CCI Injury and hCG Treatment

Correlation analyses demonstrated strong positive correlations in baseline plasma samples between P₄ with androstenedione (r=0.84, p<0.01); androstenedione with its metabolite T (r=0.82, p<0.01); and with corticosterone and its precursor 11-DOC (r=0.89, p<0.001), (Table 1). Sham injury obviated the significant correlations between sex steroids, but not corticosterone and its precursor 11-DOC (r=0.98, p<0.001, Table 2). hCG treatment of Sham animals was sufficient in restoring the strong positive relationship between T and androstenedione (r=0.76, p<0.05), but not between P₄ with androstenedione. hCG treatment induced two additional positive correlations between androstenedione with 11-DOC (r=0.84, p<0.01) and androstenedione with corticosterone (r=0.72, p<0.05), (Table 2). These results suggest that Sham surgery alone is sufficient to disrupt the relationship between sex steroid metabolism, a relationship that is partially reversed with hCG treatment.

TABLE 1
The relationship between the concentrations of plasma steroids in rats.
The top figure in each square is the coefficient of determination (r²) and the bottom figure is
the number of pairs that were analyzed. The results comprising the bottom left triangle are
for rats at baseline and the top right triangle are for rats from all time points: post-surgery
days (PSD)/post-injury days (PID) 1, 11, 19 and 29 for all treatment groups
(*p < 0.05, **p < 0.01, ***p < 0.001).

| | | All Groups and Time Points | | | | |
|---|---|---|---|---|---|---|
| | | (PSD/PID 1, 11, 19 &29) | | | | |
| | | | | Andro- | 11- | Cortico- |
| | | T | P₄ | stenedione | DOC | sterone |
| Baseline | T | | 0.13 | 0.68** * | 0.22 | 0.15 |
| Samples | | | 104 | 92 | 104 | 104 |
| (PSD/ | P₄ | 0.41 | | 0.28* | 0.29* | 0.25* |
| PID − 10) | | 10 | | 92 | 104 | 104 |
| | Andro. | 0.82** | 0.84** | | 0.33** | 0.19 |
| | | 10 | 10 | | 92 | 108 |
| | 11-DOC | 0.40 | −0.15 | 0.06 | | 0.90*** |
| | | 10 | 10 | 10 | | 104 |
| | Cortico. | 0.27 | −0.28 | −0.11 | 0.89** | |
| | | 10 | 10 | 10 | 10 | |

TABLE 2
The relationship between the concentrations of plasma steroids in Sham
surgery rats treated with or without hCG. The top figure in each square is the coefficient
of determination (r²) and the bottom figure is the number of pairs that were analyzed. The
results comprising the bottom left triangle are for Sham surgery rats treated with hCG and the
top right triangle are for Sham surgery rats treated with saline, from post-surgery days (PSD)
1, 11, 19 and 29 (*p < 0.05, **p < 0.01, ***p < 0.001).

| | | Sham + Saline (PSD 1, 11, 19 & 29) | | | | |
|---|---|---|---|---|---|---|
| | | | | Andro- | | Cortico- |
| | | T | P₄ | stenedione | 11-DOC | sterone |
| Sham + | T | | 0.29 | 0.04 | <0.01 | 0.08 |
| hCG | | | 20 | 20 | 20 | 20 |
| (PSD | P₄ | 0.05 | | 0.11 | 0.84*** | 0.87*** |
| 1, 11, 19 | | 20 | | 20 | 20 | 20 |
| & 29) | Andro. | 0.76* | 0.29 | | 0.34 | 0.34 |
| | | 20 | 14 | | 20 | 20 |
| | 11- | 0.44 | 0.27 | 0.84** | | 0.98*** |
| | DOC | 20 | 20 | 14 | | 20 |
| | Cortico. | 0.25 | 0.24 | 0.72* | 0.94*** | |
| | | 14 | 20 | 14 | 20 | |

CCI injury, like sham injury, resulted in positive relationships between P₄ with corticosterone (r=0.58, p<0.01) and 11-DOC (r=0.68, p<0.001), between corticosterone and its precursor 11-DOC (r=0.91, p<0.001, Table 3), as well as the loss of significant correlations between sex steroids. Unlike sham injury, CCI injury resulted in a strong correlation between androstenedione with its metabolite T (r=0.97, p<0.001). Like Sham animals, hCG treatment of CCI animals restored the positive relationship between T and androstenedione (r=0.64, p<0.001), while the positive correlation between corticosterone and 11-DOC (r=0.93, p<0.001) was maintained. Together, these results suggest that like Sham surgery, CCI injury disrupts sex steroid metabolism, while hCG treatment partially restores sex steroid metabolism.

TABLE 3
The relationship between the concentrations of plasma steroids in CCI
injured rats treated with or without hCG. The top figure in each square is the coefficient
of determination (r²) and the bottom figure is the number of pairs that were analyzed. The
results comprising the bottom left triangle are for CCI injury rats treated with hCG and the
top right triangle are for CCI injury rats treated with saline, from post-injury days (PID) 1,
11, 19 and 29 (*p < 0.05, **p < 0.01, ***p < 0.001).

| | | CCI + Saline (PID 1, 11, 19 & 29) | | | | |
|---|---|---|---|---|---|---|
| | | | | Andro- | | Cortico- |
| | | T | P₄ | stenedione | 11-DOC | sterone |
| CCI | T | | 0.04 | 0.97*** | −0.06 | −0.10 |
| + | | | 32 | 32 | 32 | 32 |
| hCG | P₄ | 0.27 | | 0.08 | 0.67*** | 0.58** |
| (PID | | 32 | | 32 | 32 | 32 |
| 1, 11, | Andro. | 0.64*** | 0.30 | | −0.03 | −0.08 |
| 19 & | | 32 | 32 | | 32 | 32 |
| 29) | 11- | 0.26 | 0.30 | 0.20 | | 0.91*** |
| | DOC | 32 | 32 | 32 | | 32 |
| | Cortico. | 0.32 | 0.31 | 0.20 | 0.93*** | |
| | | 32 | 32 | 32 | 32 | |

### Discussion for Examples 1-4

We demonstrate for the first time that intraperitoneal injection of hCG is effective in reversing hypogonadism (FIGS. 1A and 1B) and attenuating hypoadrenalism (FIGS. 1C and D) following a craniectomy or craniectomy+CCI injury in young adult male rats. Both craniectomy, and craniectomy+CCI injury, promoted corticosteroid production in favor of sex steroid (P₄) production, a relationship that was reversed with hCG treatment (FIGS. 1A-1D; Tables 1-3). hCG's ability to increase sex steroid plasma concentrations following a craniectomy and following a moderate-to-severe brain injury supports its potential as a treatment for TBI-induced hypogonadism.

It is important to note that while hCG has potential to reverse hypogonadism and promote neurogenesis and cognitive recovery, an increase in circulating hCG/LH concentrations as a result of ovariectomy or treatment has been shown to impair cognition in rodents, while lowering LH or blocking LHCGR signaling is protective of memory in rodents and humans. Conversely, it has been demonstrated that intracerebroventricular hCG delivery after OVX rescued dendritic spine density and spatial memory. The general negative impact of LH/hCG on cognitive performance appears to be dependent upon the ratio of gonadotropins to sex steroids since situations where gonadotropins and sex steroids are in balance such as during the adult reproductive period are periods of normal cognitive performance and do not involve dyotic signaling. This is illustrated by the findings that interventions that reverse dyotic signaling such as sex steroid supplementation of ovariectomized animals (see references above), GnRH agonist suppression of gonadotropins in post-menopausal women, and caloric restriction, either reverse or halt cognitive decline. Therefore, functioning gonads may be essential for hCG to promote cognitive recovery from a TBI, as have been found in intact male rats (unpublished data). Thus, hCG treatment might be expected to be most beneficial in pre-menopausal and pre-andropausal individuals, while those further along the post-reproduction spectrum might benefit most from a combination therapy of hCG supplemented with appropriate sex steroids.

### Causes of Craniectomy and CCI Injury Induced Hypogonadism and Hypoadrenalism

The induction of hypogonadism and hypoadrenalism in young male rats following a craniectomy and a craniectomy+CCI injury (reduction in plasma concentrations of P₄, T, 11-DOC and corticosterone; FIGS. 1A-1D) is consistent with previous reports in rats. Since hCG reversed hypogonadism, and diminished hypoadrenalism in Sprague-Dawley rats (FIGS. 1A-1D and 2A-2D; Tables 1-3), our results suggest isoflurane and/or surgical trauma/stress to the HP are impacting the long-term hypothalamic release of GnRH or the pituitary release of gonadotropins rather than the production of steroids by the testes (or adrenals). Circulating cortisol concentrations are elevated following neurosurgical procedures in humans, but not during anesthesia (nitrous oxide and halothane, after thiopentone induction). Alternatively, or in conjunction, isoflurane anesthesia administered during the craniectomy may have inhibited hypothalamic and/or pituitary function since it has been reported that isoflurane anesthesia can dose-dependently suppress circulating follicle-stimulating hormone (FSH) and T concentrations, post-natal neurogenesis, and cognitive performance in adult male Sprague-Dawley rats. Anesthesia-induced hypogonadism and hypoadrenalism represents another complication of anesthesia that could impact the recovery from and quality of life for those undergoing anesthesia for a surgical procedure. Further research is required to delineate whether this effect is attributed to isoflurane on the functioning of the hypothalamus and/or pituitary, a combination of isoflurane and surgical stress, or an effect of isoflurane early and of surgical stress later in the maintenance of the hypogonadism over 29 days.

Our data is consistent with early studies in humans demonstrating that TBI could alter hypothalamic morphology and induce hypogonadism and hypothyroidism. However, while our study implicates isoflurane in the induction of hypogonadism and hypopituitarism via suppression of HP function, human studies suggest that TBI-induced hypogonadism and hypopituitarism is mediated via suppression of HP function by elevated circulating cortisol. Ranganathan et al., (see Ranganathan P, et al., Brain Inj. 30(4): 452-461(2016)) demonstrated that the stress of TBI results in anovulation and central hypothalamic-pituitary-ovarian axis suppression, with menstruation resuming among pre-menopausal women when serum cortisol normalized to luteal phase control levels. It is apparent that TBI-induced HH, even when limited to the anterior hypothalamus, is a system problem commonly involving both the HPG and HPA axis's. From our study it is not possible to determine if the CCI injury had an impact beyond that of craniectomy on promoting hypogonadism or hypoadrenalism, as has been reported for human TBI. These results demonstrate that future studies need to take into account the effects of isoflurane alone in any model of TBI-induced hypogonadism.

### hCG Treatment or Reversing Hypopitnitarism

Our results in craniectomized and craniectomized and CCI-injured rats demonstrate that post-surgery and post-injury male rats retain the capacity to synthesize and secrete testosterone (FIGS. 1A-1D). The reversal of hypothalamic/pituitary function in animals induced by a TBI, craniectomy and/or isoflurane anesthesia indicates the utility of hCG for reversing hypogonadism and hypoadrenalism in these conditions. hCG treatment comes with the advantage of not only increasing neurotropic hCG/hLH, but the dozens of gonadal sex steroid and protein hormones that regulate normal brain structure and function.

hCG has been shown to increase testosterone production in aged male rats. hCG is a safe, cheap, FDA approved treatment for hypogonadism in men (chronically), infertility in men and women, and to promote the descent of testicles in young boys with cryptorchidism. In this context, hCG treatment has recently been shown to be effective in (1) raising plasma T concentrations in healthy men with chronic spinal cord injury, and this was not significantly different from hCG's elevation of plasma T in able-bodied male control subjects, (2) protecting the rodent adult and neonatal brain from hypoxic-ischemic cellular degeneration in vivo and inhibiting glutamate-dependent excitotoxic or necrotic neuronal cell death in vitro, and (3) increasing ERK phosphorylation, neurite outgrowth and rescuing ovariectomy-induced spatial memory deficits in C57B1/6 J mice. In addition, hCG also partially attenuated hypoadrenalism in male rats. Although there are few studies that have assessed the impact of hCG on regulating adrenal steroid production, hCG has been demonstrated to increase follicular fluid concentrations of 11-DOC, but not corticosterone, while LEβ overexpressing female mice have enlarged adrenals, increased LHCGR expression and a 14-fold elevation in serum corticosterone. In this latter study, the authors proposed that enhanced ovarian estrogen synthesis causes increased secretion of prolactin, which elevates LHCGR expression in the mouse adrenal cortex, leading to elevated, LH-dependent, corticosterone production. Continuous exposure to hCG is however known to suppress the expression of LHCGR via the down-regulation of mRNA. To circumvent the down-regulation of the receptor, in our study hCG was administered in the form of Pregnyl every other day, as is used clinically. Since initial phase half-life of urinary-derived Pregnyl is between 5.6 and 11 hours (https://www.merck.ca/), the 48 hours between doses appears sufficient to maintain LHCGR expression, as circulating concentrations of sex steroids (FIGS. 1A and 1B) were sustained over the 29-day experiment.

### RU-486 Impact on Plasma Steroid Concentrations

Elevations in circulating corticosterone observed in our study following RU-486 treatment is consistent with elevations in corticosterone in the male rats and cortisol, corticotropin or adrenocorticotropic hormone (ACTH) that is observed in human men, women, and non-human primates (Macaca fascicularis). Blocking glucocorticoid and P₄ signaling using RU-486 had little effect on sex steroid changes induced by craniectomy (uninjured) or CCI-injury, but significantly diminished the decline in 11-DOC and corticosterone concentrations in craniectomy (uninjured) but not CCI injured animals (FIGS. 2A-2D), indicating that blocking glucocorticoid (and perhaps P₄) receptor signaling partially prevents the suppression of 11-DOC and corticosterone (either by limiting stress induced suppression of 11-DOC/corticosterone or elevating their synthesis).

### Example 5: hCG Induces the Proliferation of Embryonic Stem Cells and Their Differentiation into Neuronal Precursor Cells

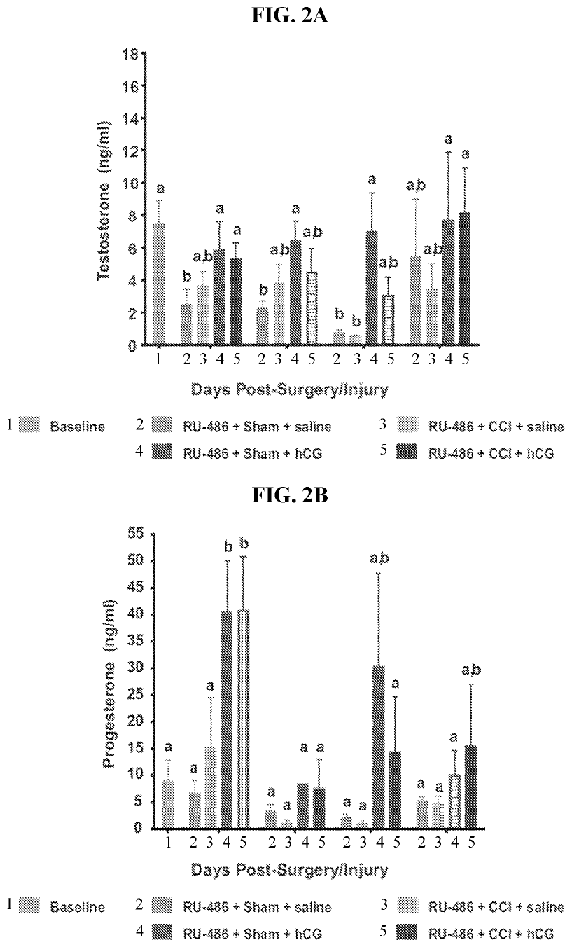

hCG is the first pregnancy hormone induced following conception (pregnancy urine test strips detect hCG). In our studies aimed at identifying the hormonal factor(s) that drive zygotic cell division in the early pre-implantation embryo, we found that in using human embryonic stem cells (hESC) as an in vitro model of early embryogenesis, hESCs express the full-length mature LH/hCG receptor (LHCGR; 92 kDa); the autocrine production of LH/hCG by hESC signals via its receptor the division of pluripotent hESC; and that inhibition of LHCGR signaling with P-antisense oligonucleotides suppresses hESC division, as does a specific blocking antibody against the extracellular activation site of LHCGR, an effect that was reversed by treatment with hCG (FIG. 3). The production of hCG by the corpus luteum, pre-implantation embryo and later the placenta is therefore a key developmental signal required for cell proliferation during embryogenesis (and beyond).

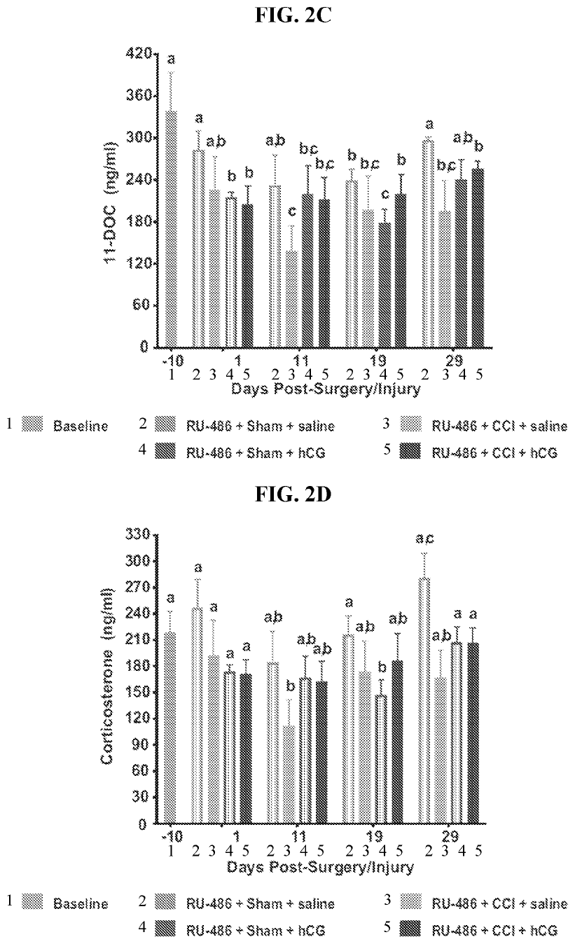

hCG, like LH, is the main regulator of sex steroid synthesis, inducing the production of P₄ which is converted into androstenedione/T and finally E₂. Not surprisingly, a physiological concentration of hCG markedly increased the secretion of P₄ from hESC (FIG. 4).

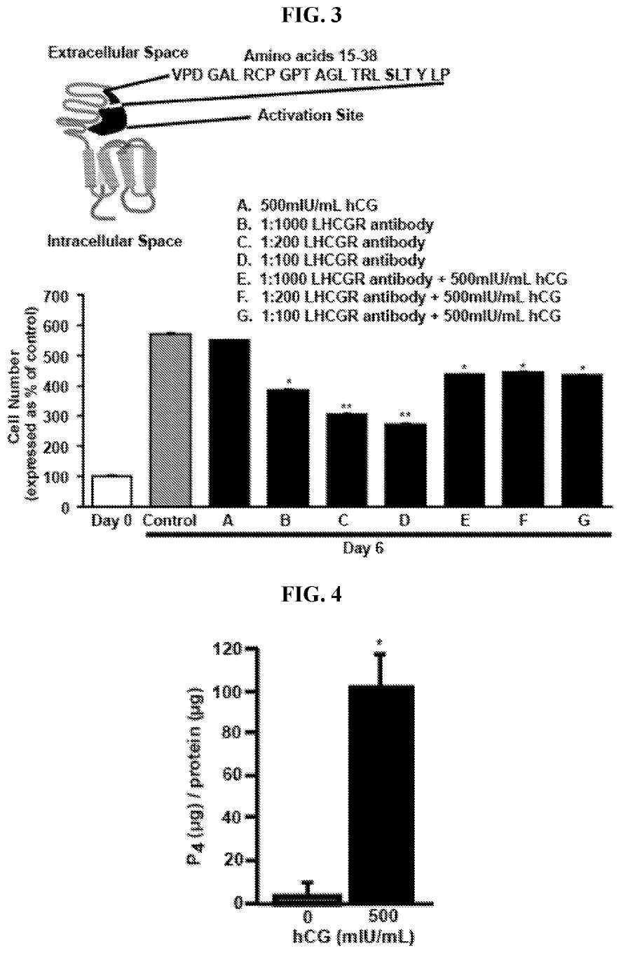

Our studies also have demonstrated that hCG-induced P₄ synthesis is obligatory for the formation of neural precursor cells (NPC) from pluripotent hESCs. hESC can be differentiated into primitive neuroectodermal (or neural precursor) cells at ˜day 10 and then into neuroectodermal cells that exhibit neural tube-like rosettes at 14-17 days of differentiation in a chemically defined neural induction media (FIGS. 5A-5D). These structures are predominantly composed of NPCs (NSCs) akin to those that form the neural tube and can be further differentiated into various neural lineages. To understand which hormonal signals regulate the specification of hESC into NSCs, we closely examined the composition of the medias and supplements that had been developed by trial and error for the maintenance of hESC, and their differentiation first into embryoid bodies (EBs) and then into NPC. We identified P₄ in the B27 supplement as being necessary and sufficient for the survival and differentiation of late embryonic neurons in vitro (E₁₈ primary hippocampal/cortical/striatal neurons). We therefore tested whether P₄ was essential for the induction of NPC from hESC. hESC were treated with P₄, E₂ (which upregulates P₄ receptor (PR) expression), or a combination of E₂+P₄. These steroids significantly decreased the rate of hESC proliferation 29%, 16% and 23%, respectively, compared to untreated control cells (FIG. 5A), suggesting they may be differentiating hESC. A screen of germline markers indicated P₄ signaling via hESC P₄ receptors increased the neuroectodermal marker nestin, an early marker of NPC (FIG. 5B). E₂ also induced nestin expression (205- and 220-kDa variants), albeit at a lower level, perhaps as a result of E₂-induced PR expression and PR signaling from endogenous P₄ production.

We next examined the morphology of the steroid treated hESC aggregates. Typically, in the presence of P₄, control rosettes display a minimum of three rosette structures inside of the cystic cavities of the hESC structures (FIG. 5C(i)). hESC grown in specially formulated media containing all the usual neural induction components with the exception of P₄ did not form neuroectodermal rosettes inside the cystic cavities of the structures (FIG. 5C(ii)). To confirm the requirement for sex steroids in the induction of NPC, hESC were treated with the PR antagonist, RU-486 just prior to entering the EB stage. No rosettes were detected in any structure treated with RU-486 (FIG. 5C(iii)). The absence of nestin in PR-antagonist treated pre-EB structures (FIG. 5D) confirmed the requirement for P₄ signaling for NPC formation. These results indicate that P₄ is essential for the differentiation of hESC into NPC. Moreover, progestagens have been shown to significantly increase rat neuroprogenitor cell and human NSC proliferation, and promote neurite development and migration that lead to changes in synaptogenesis. Importantly, P₄ enhances learning and memory in tasks mediated by the prefrontal cortex and/or hippocampus of aged mice and ovariectomized mice; and has neuroprotective effects on both neurodegenerative and cognitive processes following TBI.

### Example 6: 2 hCG/LH Signaling of Neurogenesis in the Adult Brain

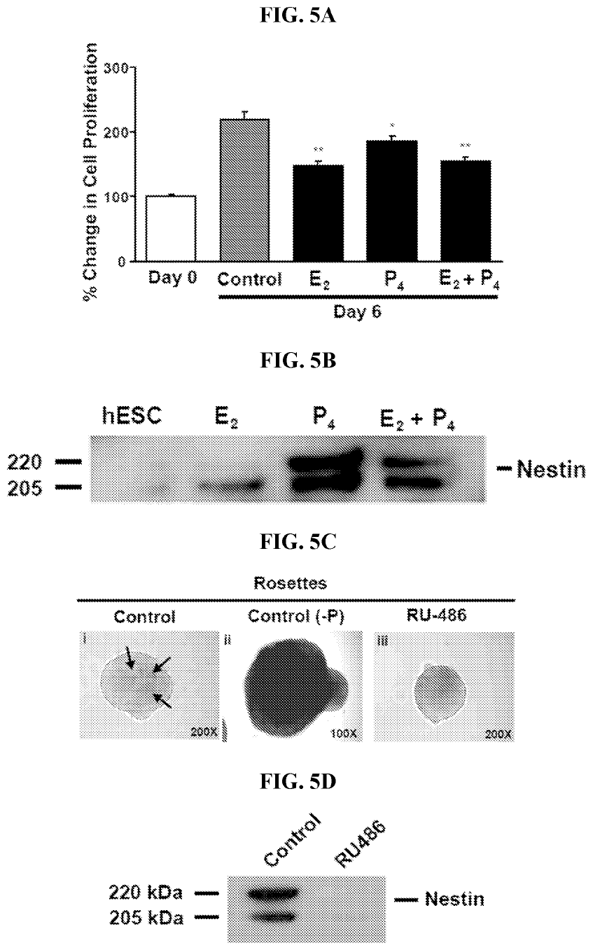

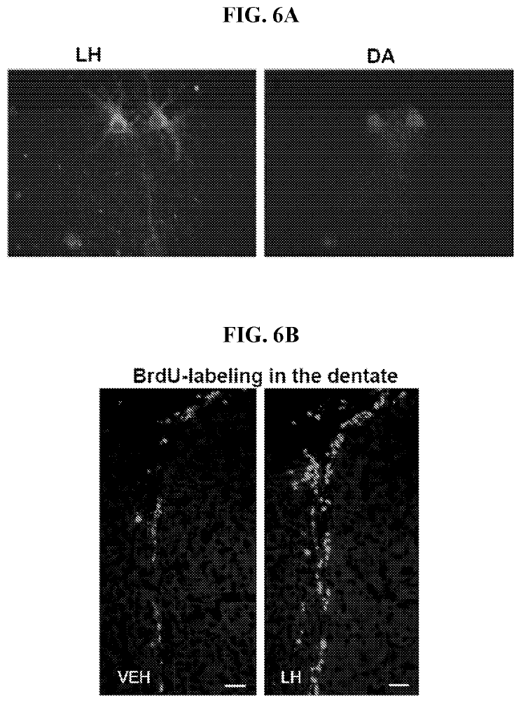

Our earlier studies demonstrated that the LH/hCG receptor (LHCGR) is expressed in the cell body and neurites of neurons (FIG. 6A), and that LH/hCG acts via neuronal LHCR to promote neurosteroid synthesis and neuron proliferation.

Mak G K, et al., Nat Neurosci 2007; 10:1003-1011 has demonstrated that LH induces cell proliferation in the dentate gyrus (DG) and subventricular zone (SVZ) of female mice, respectively. LH induced a 29% and 53% increase in the number of BrdU-labeled cells, a marker of cell proliferation, in the SVZ and dentategyrus, respectively (FIG. 6B). LH given to ovariectomized female mice also resulted in increased SVZ and dentate gyrus proliferation, supporting a direct action of LH in the dentate gyrus, rather than indirectly through estrogen release.

Together, these preliminary data indicate that gonadotropin hormones are physiological factors that signal embryonic and adult neurogenesis.

### Example 7: hCG Reverses Hypogonadotropic Hypogonadism Following a Traumatic Brain Injury

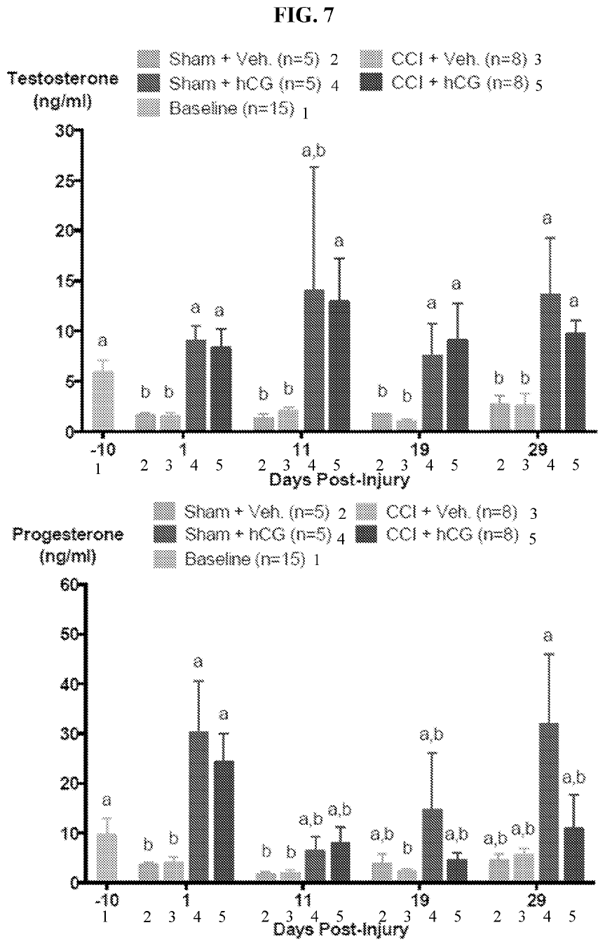

Over the last 3.5 years our lab has used controlled cortical impact (CCI) injury, a highly replicable form of focal-penetrating TBI in adult male rats, to test for neurohormones with efficacy as a treatment for TBI. The CCI to the mPFC used is moderate-severe in magnitude (injury coordinates A/P=+2.5 mm from Bregma (b); M/L=0.0 mm from midline; D/V=−3.0 to −3.5 mm from brain surface; impactor size=5 mm; velocity=2.25 m/s; dwell=100 ms; impact depth=3.5 mm). Bilateral damage to the mPFC from the CCI and retrograde degeneration of cholinergic hippocampal neurons together disrupts grip strength, locomotion, motor coordination, spatiotemporal conditioning, and increases anxiety-like behavior. To examine if this CCI injury also induced hypogonadism in our model, 5-month old adult male Sprague Dawley rats were subjected to a CCI injury to the mPFC. Sham surgery (craniectomy+saline group) in male adult rats induced a significant decline from baseline in the circulating concentrations of T (74.1%) and P₄ (63.8%) by post-injury day (PID1; FIG. 7), indicating that surgery alone was sufficient to induce HH.

To test if hCG could reverse hypogonadism following a TBI, as it has been demonstrated to do in humans, we treated Sham surgery and CCI animals with hCG (400 IU/kg/2 days) over 29 d. hCG treatment immediately reversed the hypogonadism induced by Sham surgery or CCI, increasing both T and P₄ back to post surgery concentrations, indicating that rats have the capacity to produce sex steroids at normal reproductive levels following either a craniectomy or a craniectomy+CCI. These results demonstrate the utility of hCG for reversing hypogonadism in adult male rats and suggest its use in humans following a TBI.

### Example 8: hCG Promotes Cognitive Recovery and Decreases Gross Lesion Size Following a TBI

To test the effects of hCG on cognitive and motor recovery, 69 adult male rats (5-month of age) with CCI injury (group size, n=31), sham surgery (n=24) or non-surgical control (NSCon; n=14) were immediately administered saline (n=32) or hCG (200 mIU/kg/2 days n=37) over 28 d. On PID 2, 6, 9, 13 and 23, all rats were tested for vestibulomotor performance (Rotarod) and on PID 6-10 and 20-22 the same rats were tested for learning and memory (Morris water maze (MWM)), respectively. Probe Test 1 and 2 (i.e., swimming in tank for 60 s without MWM platform) were conducted on PID 12 and 24. At the end of behavioral testing, rat brains were collected, digitally imaged and analyzed for gross lesion size (% of whole brain surface area). Note that there was considerable overlap between NSCon and Sham groups (see small standard deviations for the data) and are therefore presented as one group.

### Example 9-1: Exogenous hCG Improves Vestibulomotor Performance in CCI Adult Male Rats

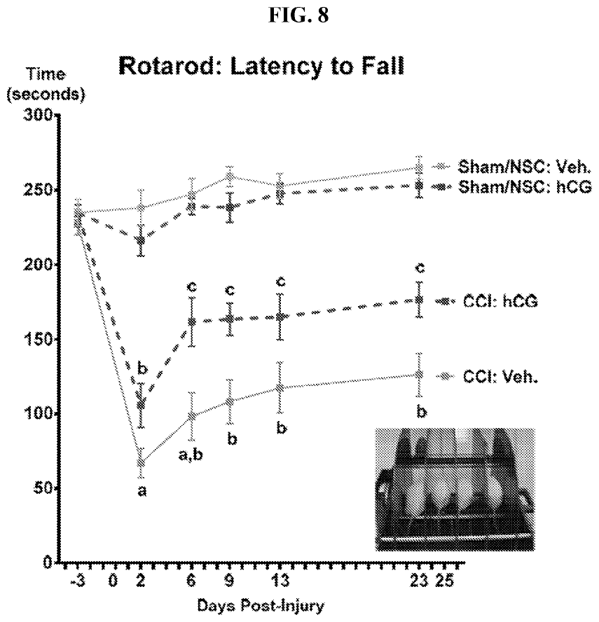

To test vestibulomotor performance, rats were trained on a Rotarod. Latency to fall from the Rotarod deceased in CCI animals as compared to the Sham groups. hCG treatment significantly improved latency to fall in CCI animals compared with vehicle treated CCI animals over the 23 d period of testing (e.g., PID23: 167.6±16.7 s vs 124±22.3 s, respectively; FIG. 8). Improvements in vestibular balance and motor coordination induced by hCG were maintained over the 23 days post-injury, indicating utility of hCG for improving vestibulomotor function.

### Example 9-2: Exogenous hCG Improves Recovery of Spatial Learning and Memory Following CCI in Adult Male Rats

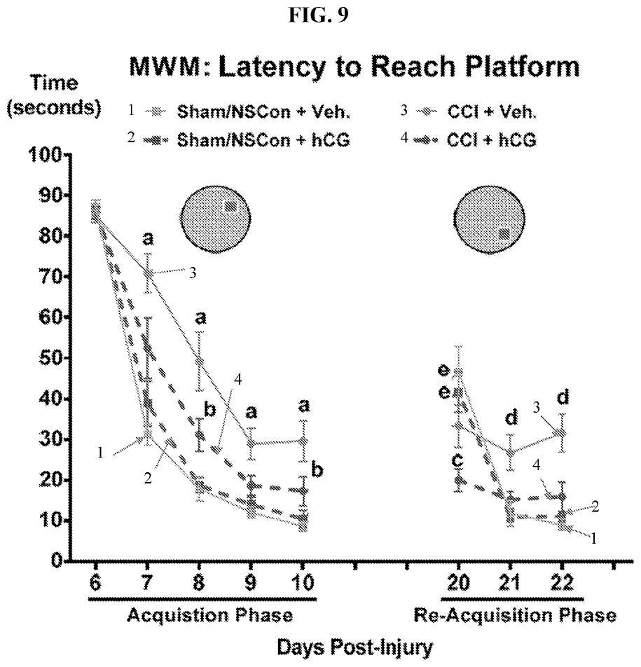

To test spatial learning and memory, rats were trained in Morris water maze performance and the latency to reach a hidden platform was recorded over 5 days (Acquisition Phase), and later, spatial memory was tested by moving the platform to a novel location (Re-acquisition Phase). We found no difference between groups in latency to reach the platform on day 1 (PID6) of the acquisition Phase (p>0.05, FIG. 9). By day 5 (PID10) of the acquisition Phase, Sham/NSCon animals treated with vehicle or hCG were fastest to find the hidden MWM platform (8.6±1.2 s to 10.4±2.1 s). Conversely, CCI animals treated with vehicle were slowest to find the hidden platform (29.5±5.0 s), while hCG significantly improved the performance of CCI animals (17.3±3.6 s) towards that of the Sham/NSCon groups by day 5 (PID10) of the acquisition phase. These results indicate that immediate and chronic hCG treatment improves spatial learning and memory in rats following a moderate-severe CCI. Ten days after the acquisition phase (PID20) the platform was moved from the NE to the SE quadrant (novel platform placement; NPP) and rats were tested for 3 days (re-acquisition phase). Time to find the hidden platform in its new location was only significantly increased in Sham/NSCon animals (FIG. 9).

After 1 NPP trial, the Sham/NSCon animals (both vehicle and hCG) quickly learned the new location of the hidden platform such that latency to find the platform improved to that at the end of the acquisition phase (Sham/NSCon vehicle: 8.6±1.2 s vs. 11.7±3.1 s; Sham/NSCon hCG: 10.4±2.1 s vs. 10.9±1.3 s, respectively). However, in the CCI groups, there was no improvement in latency to find the platform during the re-acquisition phase. In the CCI group, hCG treatment maintained the improvement in finding the platform acquired during the acquisition phase (17.3±3.6 s; PID10) throughout the re-acquisition phase (20.0±2.8 s; PID22) over that of the vehicle group (29.5±5.0 s; PID10 vs 33.3±5.3 s; PID20). These results indicate that immediate and chronic hCG improves medium-long-term spatial learning and memory in rats following a moderate-severe CCI.

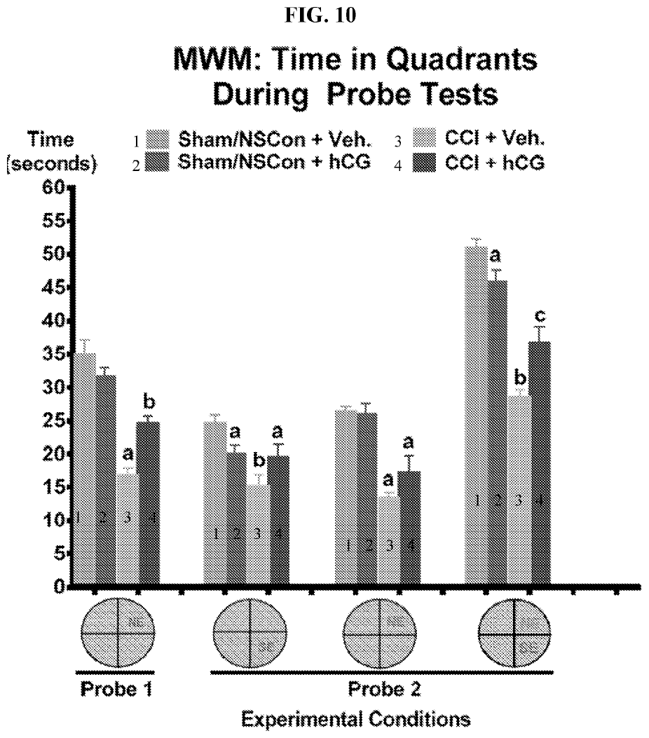

At the end of each acquisition phase, the platform was removed and 48 h later the animals were tested in the MWM for quadrant preferences over 60 s to determine if they remembered the location of the hidden platform (Probe test 1; NE quadrant). Compared with Sham/NSCon groups (34.9±2.3 s), CCI significantly decreased the amount of time spent in the NE quadrant (16.7±1.2 s) to that of chance finding (15 s), an effect that was partially reversed by hCG treatment (24.5±1.2 s; FIG. 10). This result indicates that immediate and chronic hCG improves medium-term memory following a moderate-severe CCI. Similar to Probe test 1, 48 h after completing the re-acquisition phase the platform was removed and the animals were tested for quadrant preferences (Probe test 2). CCI reduced the preference for the new SE quadrant as well as the former NE quadrant. hCG treatment significantly increased time spent in the newer SE quadrant (platform placement 2 days prior) but not in the older NE quadrant (platform placement 12 days prior). hCG also induced a small, but significant decrease in time spent in the SE quadrant by Sham/NSCon animals. Analysis of the accumulated time spent in both quadrants associated with the hidden platform indicated that compared to CCI animals treated with vehicle (28.4±1.2 s), hCG treatment improved the time spent in the platform associated quadrants (36.6±2.5 s) to that greater than chance (˜30 s). The probe tests indicate that immediate and chronic hCG promotes medium-term memory more so than long-term memory.

### Example 9-3: Exogenous hCG Reduces Gross Lesion Size Following CCI in Adult Male Rats

Post-mortem analysis of brains indicated that rats with CCI had significant tissue damage as a percent of total cortical area compared to Sham groups (10.3±1.7%; p=0.05; p<0.01), while immediate and chronic hCG treatment reduced gross lesion size by 29.1% in CCI (7.3±1.2%) compared to vehicle treated animals (data not shown).

In summary, our preliminary data indicate that hCG improves recovery of sensorimotor and spatial learning/memory dependent tasks and decreases gross lesion size in adult male Sprague Dawley rats exposed to a CCI to the mPFC (FIGS. 7-10).

### Example 10: Human Clinical Study

A male subject about 25 year old with TBI as tested by the Gleason Coma Scale (GCS) with a score of 12 or less will be administered 3 times weekly a dose of hCG (e.g., 5-200 IU/kg) for 1-18 months. The subject will be retested cognitively on a quarterly basis on the GCS (and by neuroimaging).

Separately, a post-menopausal female with TBI as tested by the Gleason Coma Scale (GCS) with a score of 12 or less will be administered 3 times weekly a dose of hCG (e.g., 5-200 IU/kg) in combination with transdermal administration of E₂ and/or P₄ (e.g., Estradiol Transdermal Patch, e.g. Vivelle/Minivelle 0.025-0.1 mg/day, or Climara 0.025 to 0.1 mg/day together with transdermal progesterone cream, e.g. ProGest, 10-40 mg/day) for 1-18 months. The subject will be retested cognitively on a quarterly basis on the GCS (and by neuroimaging).

It is anticipated that the above treated male subject or female subject would exhibit improved score in the GCS test compared to each's GCS score before the treatment or scores of controls who are not treated.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for inducing cognitive recovery of a subject suffering from a traumatic brain injury (TBI) comprising administering to the subject an effective amount of human chorionic gonadotropic (hCG), luteinizing hormone (hLH), or a combination thereof.

2. The method of claim 1, wherein the subject further suffers from a TBI-associated impairment comprising hypogonadotropic hypogonadism (HH).

3. The method of claim 2, wherein the TBI or (TBI)-associated impairment is induced by an injury to a hypothalamus or pituitary gland of the subject.

4. The method of claim 2, wherein the TBI or (TBI)-associated impairment is induced by suppression of the synthesis and secretion into the bloodstream of hormones produced by the hypothalamic-pituitary-gonadal (HPG) axis in the subject.

5. The method of claim 1, wherein the hCG comprises an α subunit and a β subunit, the hLH comprises an α subunit and a β subunit, and the hCG α subunit and the LH α subunit independently comprise an amino acid sequence of at least 90% sequence identity to SEQ ID NO: 1, the β subunit of hCG comprises an amino acid sequence of at least 90% sequence identity to SEQ ID NO: 2, and the β subunit of hLH comprises an amino acid sequence of at least 90% sequence identity to SEQ ID NO: 3.

6. The method of claim 1, wherein the hCG, hLH, or combination thereof is administered about 2 days to about 7 days per week.

7. The method of claim 1, wherein the hCG, hLH, or combination thereof is administered about 3 days to about 5 days per week.

8. The method of claim 1, wherein the hCG, hLH, or combination thereof is administered every other day, every other 2 days, or every other 3 days.

9. The method of claim 1, wherein the hCG, hLH, or combination thereof is administered for about 1 week to about 6 weeks, or about 4 weeks to 5 weeks.

10. The method of claim 1, wherein the hCG, hLH, or combination thereof is administered for about 1 months to about 18 months, optionally for about 9-18 months.

11. The method of claim 1, wherein an effective amount of the hCG, hLH, or combination thereof administered comprises about 100 IU/kg to about 1000 IU/kg of the hCG, LH, or combination thereof.

12. The method of claim 1, wherein an effective amount of the hCG, hLH, or combination thereof comprises about 200 IU/kg to about 600 IU/kg of the hCG, hLH, or combination thereof.

13. The method of claim 1, wherein an effective amount of the hCG, hLH, or combination thereof administered comprises about 300 IU/kg to about 500 IU/kg of the hCG, hLH, or combination thereof.

14. The method of claim 1, further comprising administering to the subject an effective amount of 11β-[p-(Dimethylamino)phenyl]-17α-(1-propynyl)estra-4,9-dien-17β-ol-3-one (RU-486).

15. The method of claim 1, wherein the subject is capable of normal adult gonadal hormone synthesis and secretion.

16. The method of claim 1, wherein the subject is a human, optionally wherein the human is a pre-menopausal female or a pre-andropausal male, and optionally wherein the human is a 15- to 45-year-old female or a 16- to 50-year-old male.

17. The method of claim 1, wherein the subject is not capable of normal adult gonadal hormone synthesis and secretion, and wherein the method further comprises separately, simultaneously, or subsequently administering to the subject an effective amount of a gonadal hormone.

18. The method of claim 17, wherein the gonadal hormone is selected from progesterone, estradiol, testosterone, inhibin B, Anti-Müllerian hormone (AMH), or a combination of any two or more thereof.

19. A method for inducing cognitive recovery of a subject suffering from a traumatic brain injury (TBI) comprising:
determining that the subject is capable of normal adult gonadal hormone synthesis and secretion; and
administering to the subject an effective amount of human chorionic gonadotropic (hCG), luteinizing hormone (hLH), or a combination thereof.

20. A method for inducing cognitive recovery of a subject suffering from a traumatic brain injury (TBI) comprising:
determining that the subject is not capable of normal adult gonadal hormone synthesis and secretion; and
administering to the subject an effective amount of human chorionic gonadotropic (hCG), human luteinizing hormone (hLH), or a combination thereof, and an effective amount of a gonadal hormone, optionally wherein the gonadal hormone is selected from progesterone, estradiol, testosterone, inhibin B, AMH, or a combination of any two or more thereof.
